(19) United States
(12) Patent Application Publication
(10) Pub. No.: US 2018/0353515 A1
(43) Pub. Date: Dec. 13, 2018

(54) TREATMENT OF CANCER WITH A PI3K INHIBITOR IN A PATIENT PRESSELECTED FOR HAVING A PIK3CA MUTATION IN THE CTDNA

(71) Applicant: Novartis AG, Basel (CH)

(72) Inventor: Emmanuelle di Tomaso, Lexington, MA (US)

(21) Appl. No.: 15/779,873

(22) PCT Filed: Nov. 30, 2016
(86) PCT No.: PCT/IB2016/057208

§ 371 Date: May 30, 2018

Related U.S. Application Data
(60) Provisional application No. 62/262,620, filed on Dec. 3, 2015.

(51) Int. Cl. A61K 31/5377; A61K 31/565; C12Q 1/6886

(52) U.S. Cl. CPC A61K 31/5377; A61K 31/565; C12Q 2600/156; C12Q 2600/106; C12Q 1/6886

(57) ABSTRACT

Selective cancer treatment regimes based on assaying for the presence or absence of a mutation in PI3K in a blood or serum sample obtained from a patient having cancer. The cancer is treated with 5-(2,6-di-mor-pholin-4-yl-pyrimidin-4-yl)-4-trifluoromethyl-pyridin-2-ylamine or its hydrochloride salt, or (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) on the basis that the patient is determined to have in their ctDNA a PIK3CA mutation.

### FIELD OF THE INVENTION

The present invention relates to novel personalized therapies, kits, transmittable forms of information and methods for use in treating patients having cancer.

### BACKGROUND OF THE INVENTION

Phosphatidylinositol 3-kinases (PI-3 kinase or PI3K) comprise a family of lipid and serine/threonine kinases that catalyze the transfer of phosphate to the D-3′ position of inositol lipids to produce phosphoinositol-3-phosphate (PIP), phosphoinositol-3,4-diphosphate (PIP2) and phosphoinositol-3,4,5-triphosphate (PIP3) that, in turn, act as second messengers in signaling cascades by docking proteins containing pleckstrin-homology, FYVE, Phox and other phospholipid-binding domains into a variety of signaling complexes often at the plasma membrane ((Vanhaesebroeck et al., Annu. Rev. Biochem 70:535 (2001); Katso et al., Annu. Rev. Cell Dev. Biol. 17:615 (2001)). Of the two Class 1 PI3Ks, Class 1A PI3Ks are heterodimers composed of a catalytic p110 subunit (α, β, δ isoforms) constitutively associated with a regulatory subunit that can be p85α, p55α, p50α, p85β or p55γ. The Class IB sub-class has one family member, a heterodimer composed of a catalytic p110γ subunit associated with one of two regulatory subunits, p101 or p84 (Fruman et al., Annu Rev. Biochem. 67:481 (1998); Suire et al., Curr. Biol. 15:566 (2005)). The modular domains of the p85/55/50 subunits include Src Homology (SH2) domains that bind phosphotyrosine residues in a specific sequence context on activated receptor and cytoplasmic tyrosine kinases, resulting in activation and localization of Class 1A PI3Ks. Class IB PI3K is activated directly by G protein-coupled receptors that bind a diverse repertoire of peptide and non-peptide ligands (Stephens et al., Cell 89:105 (1997)); Katso et al., Annu. Rev. Cell Dev. Biol. 17:615-675 (2001)). Consequently, the resultant phospholipid products of class I PI3K link upstream receptors with downstream cellular activities including proliferation, survival, chemotaxis, cellular trafficking, motility, metabolism, inflammatory and allergic responses, transcription and translation (Cantley et al., Cell 64:281 (1991); Escobedo and Williams, Nature 335:85 (1988); Fantl et al., Cell 69:413 (1992)).

PI-3 kinase inhibitors are useful therapeutic compounds for the treatment of various conditions in humans. Aberrant regulation of PI3K, which often increases survival through Akt activation, is one of the most prevalent events in human cancer and has been shown to occur at multiple levels. In some tumors, the genes for the p110a isoform, PIK3CA, are amplified and increased protein expression of their gene products has been demonstrated in several human cancers. In other tumors, somatic missense mutations in PIK3CA that activate downstream signaling pathways have been described at significant frequencies in a wide diversity of human cancers (Kang et al., Proc. Natl. Acad. Sci. USA 102:802 (2005); Samuels et al., Science 304:554 (2004); Samuels et al., Cancer Cell 7:561-573(2005)). Deregulation of phosphoinositol-3 kinase is a common deregulation associated with human cancers and proliferative diseases.

The specific pyrimidine derivative compound of formula (II)

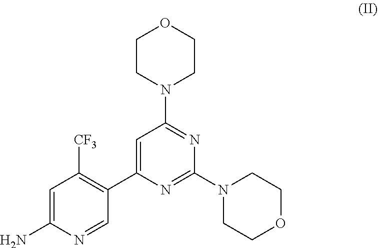 [SMILES: Nc1cc(C(F)(F)F)c(-c2cc(N3CCOCC3)nc(N3CCOCC3)n2)cn1]

and its pharmaceutically acceptable salts are pan-PI3K inhibitors which may be used for the treatment of cancer. The compound of formula (II) has the chemical name 5-(2,6-di-morpholin-4-yl-pyrimidin-4-yl)-4-trifluoromethyl-pyridin-2-ylamine. This compound and its preparation are disclosed in WO2007/084786. Such pyrimidine derivative is proven to be an effective PI3K inhibitor, e.g. WO2007/084786 and S. Maira et al, Molecular Cancer Therapeutics 11:317-328 (2012), that displays broad activity against a large panel of cultured human cancer cell lines.

There is an increasing body of evidence that suggests a patient's genetic profile can be determinative to a patient's responsiveness to a therapeutic treatment. Given the numerous therapies available to an individual having cancer, a determination of the genetic factors that influence, for example, response to a particular drug, could be used to provide a patient with a personalized treatment regime. Such personalized treatment regimes offer the potential to maximize therapeutic benefit to the patient while minimizing related side effects that can be associated with alternative and less effective treatment regimes. Thus, there is a need to identify factors which can be used to predict whether a patient is likely to respond to a particular therapeutic therapy.

### SUMMARY OF THE INVENTION

The present invention is based on the finding that the presence of a PIK3CA mutation in circulating tumor DNA of patients with cancer is predictive that such patients are more likely to respond to a PI3K inhibitor selected from the group consisting of 5-(2,6-di-morpholin-4-yl-pyrimidin-4-yl)-4-trifluoromethyl-pyridin-2-ylamine and its hydrochloride salt and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide), particularly 5-(2,6-di-morpholin-4-yl-pyrimidin-4-yl)-4-trifluoromethyl-pyridin-2-ylamine or its hydrochloride salt.

In one aspect, the invention includes a method of treating a patient having a cancer, comprising administering a therapeutically effective amount of a PI3K inhibitor selected from the group consisting of 5-(2,6-di-morpholin-4-yl-pyrimidin-4-yl)-4-trifluoromethyl-pyridin-2-ylamine and its hydrochloride salt and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl})-amide) to the patient on the basis of the patient having been determined to have in their circulating tumor DNA (ctDNA) a PIK3CA mutation. In one example, the method can include administering a therapeutically effective amount of a PI3K inhibitor selected from the group consisting of 5-(2,6-di-morpholin-4-yl-pyrimidin-4-yl)-4-trifluoromethyl-pyridin-2-ylamine and its hydrochloride salt and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) to the patient on the basis of the patient having been determined to have in their ctDNA a PIK3CA mutation; or alternatively, administering a therapeutically effective amount of a therapeutic other than a PI3K inhibitor selected from the group consisting of 5-(2,6-di-morpholin-4-yl-pyrimidin-4-yl)-4-trifluoromethyl-pyridin-2-ylamine and its hydrochloride salt and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) to the patient on the basis of the patient not having been determined to have in their ctDNA a PIK3CA mutation.

Examples of a therapeutic other than a PI3K inhibitor selected from the group consisting of 5-(2,6-di-morpholin-4-yl-pyrimidin-4-yl)-4-trifluoromethyl-pyridin-2-ylamine and its hydrochloride salt and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) are fulvestrant, trastuzumab, lapatinib, gefinitib, erlotinib, paclitaxel, everolimus, methotrexate, fluorouracil, anastrozole, exemestane, capecitabine, cyclophosphamide, letrozole, toremifene, gemcitabine hydrochloride, goserelin acetate, palbociclib, megestrol acetate, tamoxifen, palbociclib, pertuzumab, or vinblastine and combinations thereof.

The method of the invention can be used to treat any cancer including a cancer of the lung and bronchus; prostate; breast; pancreas; colon and rectum; thyroid; liver and intrahepatic bile duct; hepatocellular; gastric; glioma/glioblastoma; endometrial; melanoma; kidney and renal pelvis; urinary bladder; uterine corpus; uterine cervix; ovary; head and neck; multiple myeloma; esophagus; acute myelogenous leukemia; chronic myelogenous leukemia; lymphocytic leukemia; myeloid leukemia; brain; oral cavity and pharynx; larynx; small intestine; non-Hodgkin lymphoma; melanoma; and villous colon adenoma. In one example, the cancer is selected from breast cancer and head and neck cancer. In another example, the cancer is breast cancer, such as metastatic breast cancer.

In another aspect, the invention includes a method of treating a patient having a cancer with a PI3K inhibitor selected from the group consisting of 5-(2,6-di-morpholin-4-yl-pyrimidin-4-yl)-4-trifluoromethyl-pyridin-2-ylamine and its hydrochloride salt and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide), including selecting the patient for treatment with said PI3K inhibitor on the basis of the patient having been determined to have in their circulating tumor DNA (ctDNA) a PIK3CA mutation; and thereafter, administering a therapeutically effective amount of said PI3K inhibitor to the patient.

In yet another aspect, the invention includes a method of treating a patient having a cancer with a PI3K inhibitor, including assaying a blood or a plasma sample comprising ctDNA from the patient having breast cancer for the presence of a PIK3 CA mutation in the ctDNA; and administering a therapeutically effective amount of a PI3K inhibitor selected from the group consisting of 5-(2,6-di-morpholin-4-yl-pyrimidin-4-yl)-4-trifluoromethyl-pyridin-2-ylamine and its hydrochloride salt and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl})-amide) to the patient on the basis of that patient having been determined to have a PIK3CA mutation.

The methods described above can include determining the presence of any PIK3CA mutation such as a mutation in exon 1, 2, 5, 7, 9 and/or 20 in the PIK3CA gene. In one example, the PIK3CA mutation comprises one or more of the following mutations R263Q, R277W, R278W, K331E, K333N, K333N, G353D, E1093K, C1258R, E1624K, E1633K, E1634G, Q1636K, H3140K, H3140R, H3140L, and/or H3139Y.

The method described above can be performed by detecting for the presence of the PI3KCA mutation in ctDNA by polymerase chain reaction (PCR), reverse transcription-polymerase chain reaction (RT-PCR), TaqMan-based assays, direct sequencing, or Beaming.

In one example, the 5-(2,6-di-morpholin-4-yl-pyrimidin-4-yl)-4-trifluoromethyl-pyridin-2-ylamine or its hydrochloride salt is administered orally of about 60 mg to about 120 mg per day to said patient.

In another aspect, the invention includes 5-(2,6-di-morpholin-4-yl-pyrimidin-4-yl)-4-trifluoromethyl-pyridin-2-ylamine or its hydrochloride salt for use in treating a cancer, characterized in that a therapeutically effective amount of 5-(2,6-di-morpholin-4-yl-pyrimidin-4-yl)-4-trifluoromethyl-pyridin-2-ylamine or its hydrochloride salt is administered to the patient on the basis of said patient having been determined to comprise in their circulating tumor DNA (ctDNA) a PIK3CA mutation. The therapeutically effective amount of the 5-(2,6-di-morpholin-4-yl-pyrimidin-4-yl)-4-trifluoromethyl-pyridin-2-ylamine or its hydrochloride salt is administered to the patient on the basis of said patient having one or more mutations R263Q, R277W, R278W, K331E, K333N, K333N, G353D, E1093K, C1258R, E1624K, E1633K, E1634G, Q1636K, H3140K, H3140R, H3140L, and H3139Y in the PIK3CA gene.

In another aspect, the invention includes a method of predicting the likelihood that a patient having a cancer will respond to treatment with a PI3K inhibitor selected from the group consisting of 5-(2,6-di-morpholin-4-yl-pyrimidin-4-yl)-4-trifluoromethyl-pyridin-2-ylamine and its hydrochloride salt and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide), preferably 5-(2,6-di-morpholin-4-yl-pyrimidin-4-yl)-4-trifluoromethyl-pyridin-2-ylamine or its hydrochloride salt, comprising assaying a blood or serum sample comprising a tumor cell obtained from the patient for the presence of a PIK3 CA mutation, wherein:
a) the presence of the PIK3CA mutation is indicative of an increased likelihood that the patient will respond to treatment with said PI3K inhibitor; and
b) the absence of the PIK3CA mutation is indicative of a decreased likelihood that the patient will respond to treatment with said PI3K inhibitor.

In one example, the tumor cell is a circulating tumor cell or a circulating tumor DNA. The methods of the invention can be used to treat any cancer such as lung and bronchus; prostate; breast; pancreas; colon and rectum; thyroid; liver and intrahepatic bile duct; hepatocellular; gastric; glioma/glioblastoma; endometrial; melanoma; kidney and renal pelvis; urinary bladder; uterine corpus; uterine cervix; ovary; head and neck; multiple myeloma; esophagus; acute myelogenous leukemia; chronic myelogenous leukemia; lymphocytic leukemia; myeloid leukemia; brain; oral cavity and pharynx; larynx; small intestine; non-Hodgkin lymphoma; melanoma; and villous colon adenoma. In one example, the cancer is selected from breast cancer and head and neck cancer. In another example, the cancer is breast cancer such as HR+, HER2-negative locally advanced or metastatic breast cancer. In another aspect, the invention includes a method of treating a patient having a metastatic cancer, comprising administering a therapeutically effective amount of a PI3K inhibitor selected from the group consisting of 5-(2,6-di-morpholin-4-yl-pyrimidin-4-yl)-4-trifluoromethyl-pyridin-2-ylamine and its hydrochloride salt and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide), preferably 5-(2,6-di-morpholin-4-yl-pyrimidin-4-yl)-4-trifluoromethyl-pyridin-2-ylamine or its hydrochloride salt, to the patient on the basis of the patient having been determined to have in their circulating tumor DNA (ctDNA) one or more PIK3CA mutations including R263Q, R277W, R278W, K331E, K333N, K333N, G353D, E1093K, C1258R, E1624K, E1633K, E1634G, Q1636K, H3140K, H3140R, H3140L, and H3139Y.

The term “pharmaceutically acceptable” means a nontoxic material that does not interfere with the effectiveness of the biological activity of the active ingredient(s).

The term “administering” in relation to a compound, e.g., is used to refer to delivery of that compound to a patient by any route.

As used herein, a “therapeutically effective amount” refers to an amount of a PI3K inhibitor selected from the group consisting of 5-(2,6-di-morpholin-4-yl-pyrimidin-4-yl)-4-trifluoromethyl-pyridin-2-ylamine and its hydrochloride salt and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl})-amide) that is effective, upon single or multiple dose administration to a patient (such as a human) for treating, preventing, preventing the onset of, curing, delaying, reducing the severity of, ameliorating at least one symptom of a disorder or recurring disorder, or prolonging the survival of the patient beyond that expected in the absence of such treatment. When applied to an individual active ingredient (e.g., 5-(2,6-di-morpholin-4-yl-pyrimidin-4-yl)-4-trifluoromethyl-pyridin-2-ylamine or its hydrochloride salt) administered alone, the term refers to that ingredient alone.

The term “treatment” or “treat” refer to both prophylactic or preventative treatment as well as curative or disease modifying treatment, including treatment of a patient at risk of contracting the disease or suspected to have contracted the disease as well as patients who are ill or have been diagnosed as suffering from a disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment. It is understood that the term “treatment” or “treat” may be used to specifically refer to prophylactic treatment only.

The phrase “respond to treatment” is used to mean that a patient, upon being delivered a particular treatment, e.g., 5-(2,6-di-morpholin-4-yl-pyrimidin-4-yl)-4-trifluoromethyl-pyridin-2-ylamine or its hydrochloride salt or (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide), shows a clinically meaningful benefit from said treatment. In the case of breast cancer, such benefit may be measured by a variety of criteria e.g., see Example 1 progression free survival. All such criteria are acceptable measures of whether a cancer patient is responding to a given treatment. The phrase “respond to treatment” is meant to be construed comparatively, rather than as an absolute response. For example, a patient having a PIK3CA mutation is predicted to have more benefit from treatment with 5-(2,6-di-morpholin-4-yl-pyrimidin-4-yl)-4-trifluoromethyl-pyridin-2-ylamine or its hydrochloride salt than a patient who does not have a PIK3CA mutation

The phrase “receiving data” is used to mean obtaining possession of information by any available means, e.g., orally, electronically (e.g., by electronic mail, encoded on diskette or other media), written, etc.

As used herein, “selecting” and “selected” in reference to a patient is used to mean that a particular patient is specifically chosen from a larger group of patients on the basis of (due to) the particular patient having a predetermined criteria, e.g., the patient does not have a PIK3CA mutation or the patient has a PIK3CA mutation in its ctDNA. Similarly, “selectively treating a patient having a cancer” refers to providing treatment to a cancer patient, preferably a breast cancer patient, that is specifically chosen from a larger group of patients on the basis of (due to) the particular patient having a predetermined criteria, e.g., the patient does not have PIK3CA mutation or the patient has a PIK3CA mutation. Similarly, “selectively administering” refers to administering a drug to a cancer patient that is specifically chosen from a larger group of patients on the basis of (due to) the particular patient having a predetermined criteria, e.g., a PIK3CA mutation. By selecting, selectively treating and selectively administering, it is meant that a patient is delivered a personalized therapy for a specific cancer based on the patient's biology, rather than being delivered a standard treatment regimen based solely on having said cancer.

As used herein, “predicting” indicates that the methods described herein provide information to enable a health care provider to determine the likelihood that an individual having a specific cancer, preferably breast cancer, will respond to or will respond more favorably to treatment with PI3K inhibitor. It does not refer to the ability to predict response with 100% accuracy. Instead, the skilled artisan will understand that it refers to an increased probability.

As used herein, “likelihood” and “likely” is a measurement of how probable an event is to occur. It may be used interchangably with “probability”. Likelihood refers to a probability that is more than speculation, but less than certainty. Thus, an event is likely if a reasonable person using common sense, training or experience concludes that, given the circumstances, an event is probable. In some embodiments, once likelihood has been ascertained, the patient may be treated (or treatment continued, or treatment proceed with a dosage increase) with a PI3K inhibitor selected from the group consisting of 5-(2,6-di-morpholin-4-yl-pyrimidin-4-yl)-4-trifluoromethyl-pyridin-2-ylamine and its hydrochloride salt and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl})-amide) or the patient may not be treated (or treatment discontinued, or treatment proceed with a lowered dose) with a PI3K inhibitor selected from the group consisting of 5-(2,6-di-morpholin-4-yl-pyrimidin-4-yl)-4-trifluoromethyl-pyridin-2-ylamine and its hydrochloride salt and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide).

The phrase “increased likelihood” refers to an increase in the probability that an event will occur. For example, some methods herein allow prediction of whether a patient will display an increased likelihood of responding to treatment with a PI3K inhibitor selected from the group consisting of 5-(2,6-di-morpholin-4-yl-pyrimidin-4-yl)-4-trifluoromethyl-pyridin-2-ylamine and its hydrochloride salt and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) based on that patient having been determined to have a PIK3CA mutation in blood sample, e.g., in its ctDNA.

The phrase “decreased likelihood” refers to a decrease in the probability that an event will occur. For example, the methods herein allow prediction of whether a patient will display a decreased likelihood of responding to treatment with a PI3K inhibitor selected from the group consisting of 5-(2,6-di-morpholin-4-yl-pyrimidin-4-yl)-4-trifluoromethyl-pyridin-2-ylamine and its hydrochloride salt and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) based on that patient not having been determined to have a PIK3CA mutation in its blood sample, e.g., in its ctDNA.

### DETAILED DESCRIPTION OF THE FIGURES

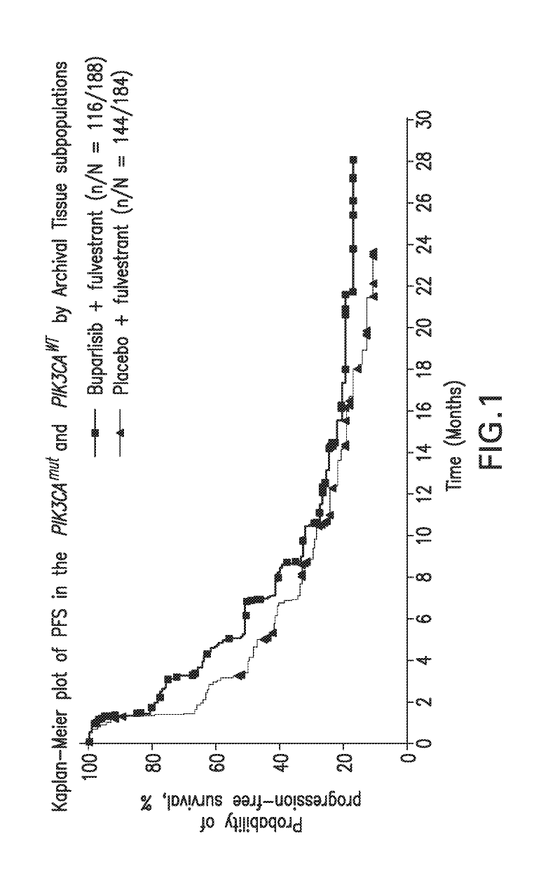

FIG. 1 shows a Kaplan-Meier plot of Progression Free-Survival (PFS) in the PIK3CA^{mut} and PIK3CA^{WT} by Archival Tissue subpopulations in Study CBKM120F2302.

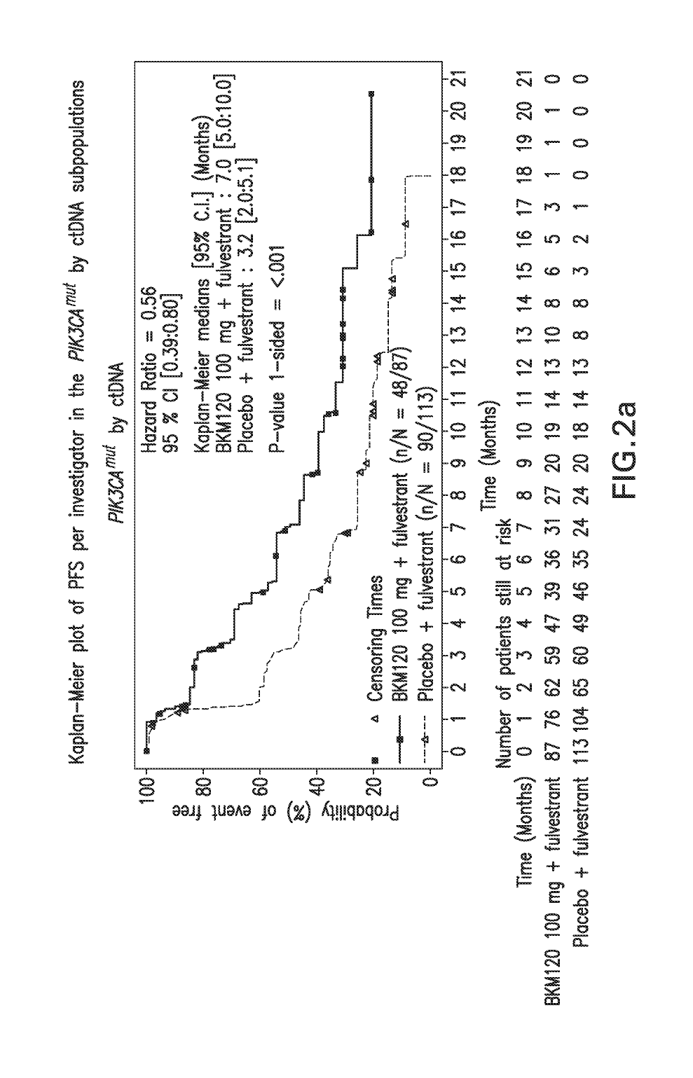

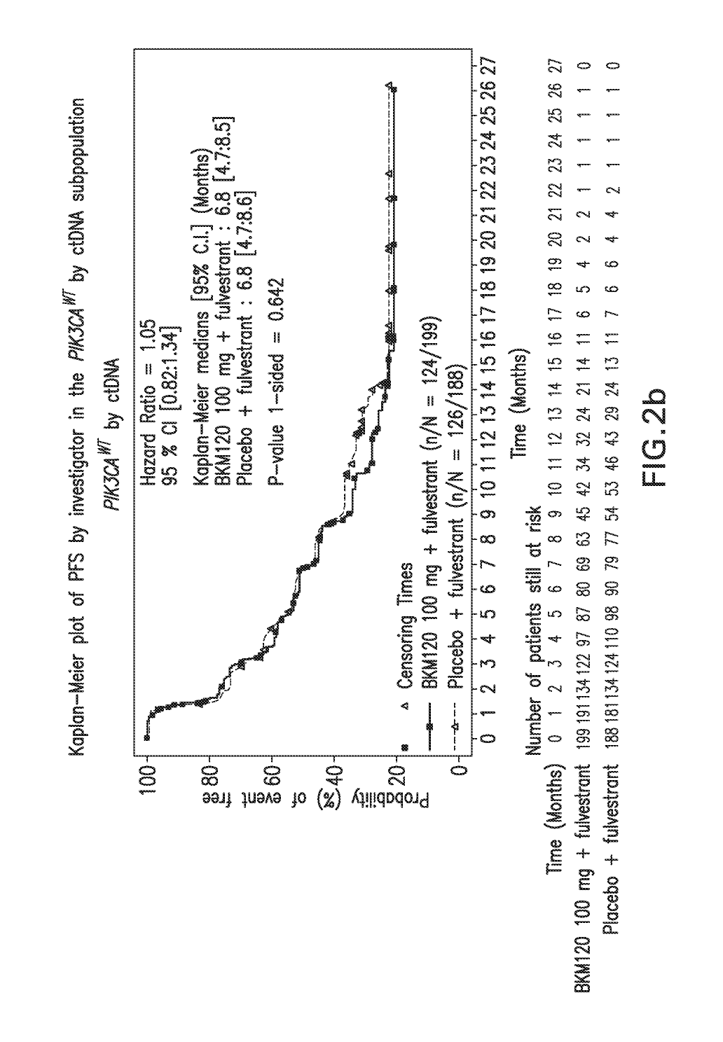

FIG. 2 shows a Kaplan-Meier plot of Progression Free-Survival (PFS) per investigator in the PIK3CA^{mut} and PIK3CA^{WT} by ctDNA subpopulations in Study CBKM120F2302.

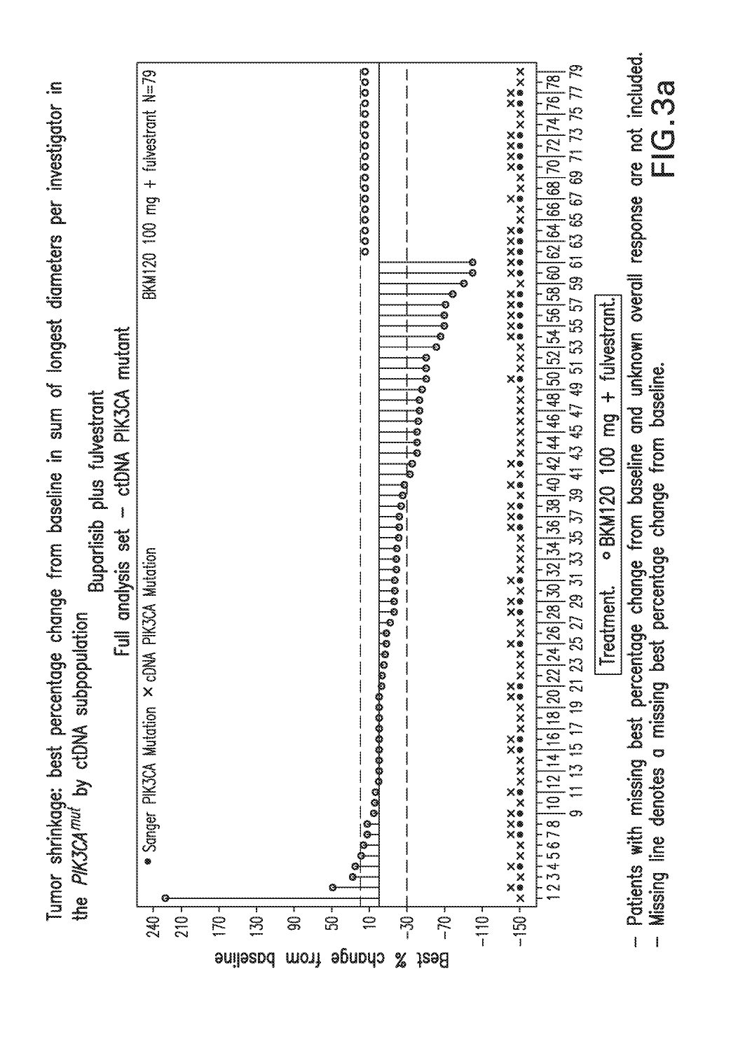

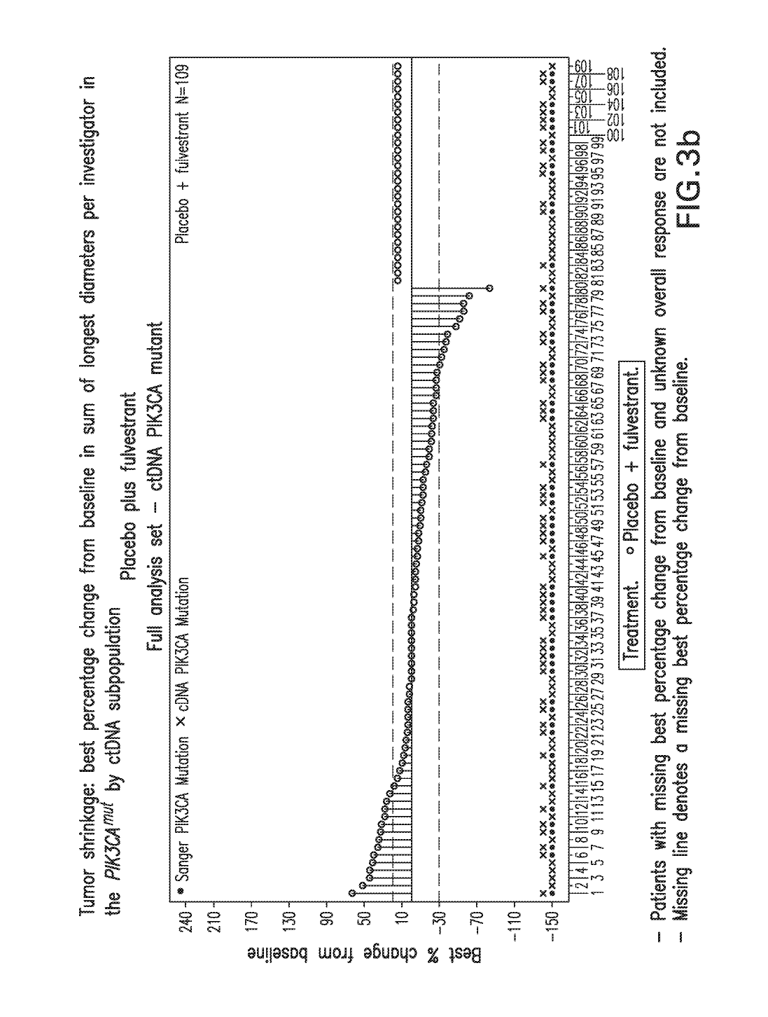

FIG. 3 shows a graph demonstrating the best percentage change from baseline in sum of longest diameters for (a) combination of buparlisib plus fulvestrant, and (b) combination of placebo plus fulvestrant per investigator in the PIK3CA^{mut} by ctDNA subpopulation in Study CBKM120F2302.

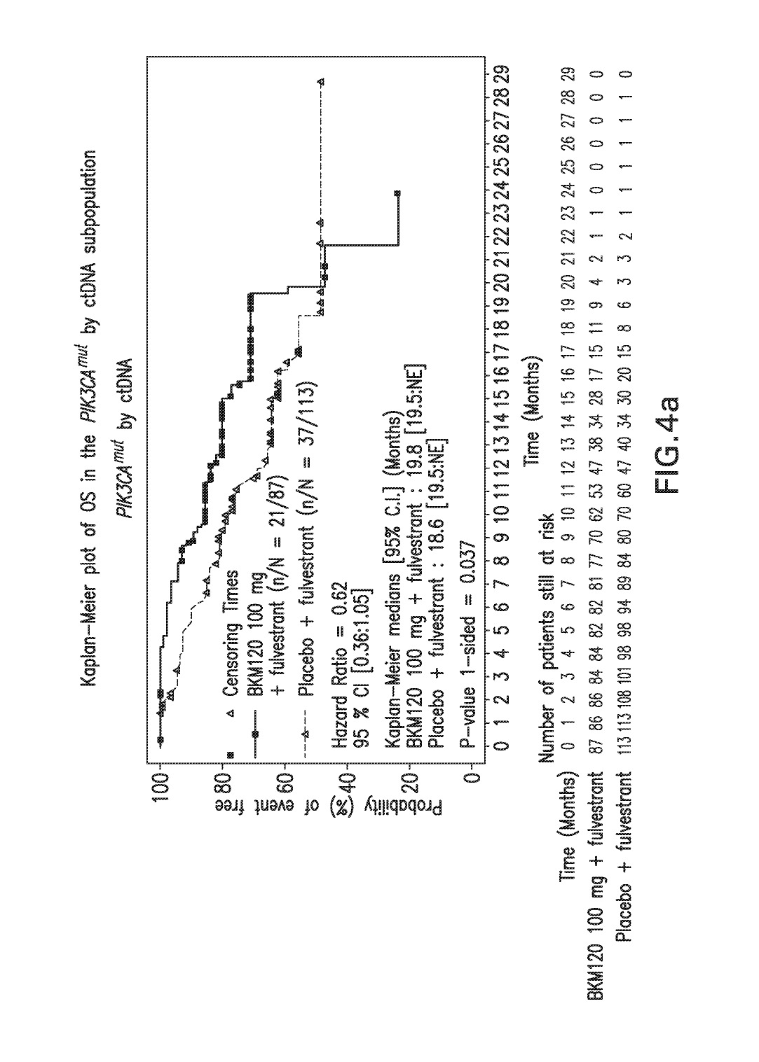

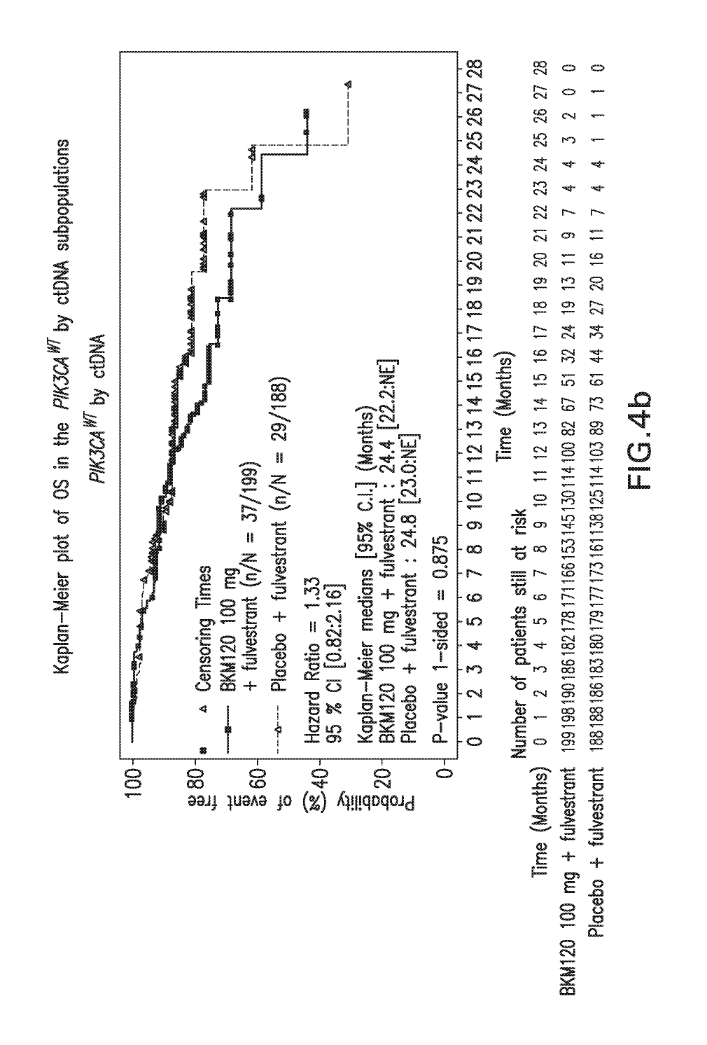

FIG. 4 shows a Kaplan-Meier plot of Overall Survival (OS) in the PIK3CA^{mut} and PIK3CA^{WT} by ctDNA subpopulations in Study CBKM120F2302.

### DETAILED DESCRIPTION OF THE INVENTION

The present invention is based on the finding that the presence or absence of a PIK3CA mutation in circulating tumor DNA (ctDNA) of a patient having a cancer, preferably breast cancer, can be used to determine the likelihood of response of a patient to therapy with a PI3K inhibitor compound. Specifically, it was found that a PIK3CA mutation in ctDNA such as a mutation in exon 9 (E545K) or exon 20 (H1047R/L) is more likely to respond to treatment with the PI3K Inhibitor 5-(2,6-di-morpholin-4-yl-pyrimidin-4-yl)-4-trifluoromethyl-pyridin-2-ylamine or its hydrochloride salt. In contrast, a nucleic acid sequence from a patient's sample not having a mutation that encodes a variant in its ctDNA, e.g., at position 545 or 1047, is less likely to respond to treatment with the PI3K inhibitor compound 5-(2,6-di-morpholin-4-yl-pyrimidin-4-yl)-4-trifluoromethyl-pyridin-2-ylamine or its hydrochloride salt. Such a patient should be treated with an alternative cancer therapy such as a chemotherapeutic or a different PI3K inhibitor (as used herein different type of PI3K inhibitor should be an inhibitor which is not 5-(2,6-di-morpholin-4-yl-pyrimidin-4-yl)-4-trifluoromethyl-pyridin-2-ylamine or its hydrochloride salt), and can be, but not limited to, treatment with a chemotherapeutic or an alternate PI3K inhibitor.

In some embodiments of the methods of the invention, the presence or absence of a PIK3 CA mutation in ctDNA may be detected by assaying for a genomic sequence or a nucleic acid product.

PI3K Inhibitors

A patient being assessed using the method disclosed herein is one who is being considered for treatment with a PI3K inhibitor. According to the present invention patients having a PIK3CA mutation in ctDNA are more likely to respond to treatment with PI3K inhibitor selected from the group consisting of 5-(2,6-di-morpholin-4-yl-pyrimidin-4-yl)-4-trifluoromethyl-pyridin-2-ylamine and its hydrochloride salt and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide), particularly the PI3K inhibitor 5-(2,6-di-morpholin-4-yl-pyrimidin-4-yl)-4-trifluoromethyl-pyridin-2-ylamine (also known as BKM120 or Compound of Formula (II) or buparlisib) or its hydrochloride salt.

PI3 kinase inhibitors can include, but are not limited to, 4-[2-(1H-Indazol-4-yl)-6-[[4-(methylsulfonyl)piperazin-1-yl]methyl]thieno[3,2-d]pyrimidin-4-yl]morpholine (also known as GDC 0941 and described in PCT Publication Nos. WO 09/036082 and WO 09/055730), 2-Methyl-2-[4-[3-methyl-2-oxo-8-(quinolin-3-yl)-2,3-dihydroimidazo[4,5-c]quinolin-1-yl]phenyl]propionitrile (also known as BEZ 235 or NVP-BEZ 235, and described in PCT Publication No. WO 06/122806), BKM120 and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) (also known as BYL719).

In one embodiment, a PI3K inhibitor is se lected from the group consisting of a compound of formula (I),

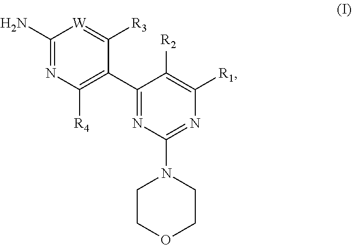

wherein

wherein W is CR_{w} or N, wherein
R_{w} is selected from the group consisting of:
(1) hydrogen,
(2) cyano,
(3) halogen,
(4) methyl,
5) trifluoromethyl,
(6) sulfonamide;
   R₁ is selected from the group consisting of:
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyl,
(8) substituted and unsubstituted aryl,
(9) substituted and unsubstituted heteroaryl,
(10) substituted and unsubstituted heterocyclyl,
(11) substituted and unsubstituted cycloalkyl,
(12) —COR₁ₐ,
(13) —CO₂R₁ₐ,
(14) —CONR₁ₐR_{1b},
(15) —NR₁ₐR_{1b},
(16) —NR₁ₐCOR_{1b},
(17) —NR₁ₐSO₂R_{1b},
(18) —OCOR₁ₐ,
(19) —OR₁ₐ,
(20) —SR₁ₐ,
(21) —SOR₁ₐ,
(23) —SO₂NR₁ₐR_{1b} wherein
   R₁ₐ, and R_{1b} are independently selected from the group consisting of:
(a) hydrogen,
(b) substituted or unsubstituted alkyl,
(c) substituted and unsubstituted aryl,
(d) substituted and unsubstituted heteroaryl,
(e) substituted and unsubstituted heterocyclyl, and
(f) substituted and unsubstituted cycloalkyl;
   R₂ is selected from the group consisting of:
(1) hydrogen,
(2) cyano,
(3) nitro,
(4) halogen,
(5) hydroxy,
(6) amino,
(7) substituted and unsubstituted alkyl,
(8) —COR₂ₐ, and
(9) —NR₂ₐCOR_{2b}, wherein
   R₂ₐ, and R_{2b} are independently selected from the group consisting of:
(a) hydrogen, and
(b) substituted or unsubstituted alkyl;
   R₃ is selected from the group consisting of:
(1) hydrogen,
(2) cyano,
(3) nitro,
(4) halogen,
(5) substituted and unsubstituted alkyl,
(6) substituted and unsubstituted alkenyl,
(7) substituted and unsubstituted alkynyl,
(8) substituted and unsubstituted aryl,
(9) substituted and unsubstituted heteroaryl,
(10) substituted and unsubstituted heterocyclyl,
(11) substituted and unsubstituted cycloalkyl,
(12) —COR₃ₐ,
(14) —NR₃ₐR_{3b},
(13) —NR₃ₐCOR_{3b},
(15) —NR₃ₐSO₂R_{3b},
(16) —OR₃ₐ,
(17) —SR₃ₐ,
(18) —SOR₃ₐ,
(19) —SO₂R₃ₐ, wherein
   R₃ₐ, and R_{3b} are independently selected from the group consisting of:
(a) hydrogen,
(b) substituted or unsubstituted alkyl,
(c) substituted and unsubstituted aryl,
(d) substituted and unsubstituted heteroaryl,
(e) substituted and unsubstituted heterocyclyl, and
(f) substituted and unsubstituted cycloalkyl; and
   R₄ is selected from the group consisting of
(1) hydrogen, and
(2) halogen.
   or a pharmaceutically acceptable salt thereof.

The radicals and symbols as used in the definition of a compound of formula (I) have meanings as disclosed in WO07/084786 which publication is hereby incorporated into the present application by reference in its entirety.

The PI3K inhibitor compound of formula (I) may be present in the form of the free base or a pharmaceutically acceptable salt thereof. Suitable salts of the compound of formula (I) include but are not limited to the following: acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemi-sulfate, heptanoate, hexanoate, fumarate, hydrochloride, hydrobromide, hydroiodide, 2 hydroxyethanesulfonate, lactate, maleate, methanesulfonate, nicotinate, 2 naphth-alenesulfonate, oxalate, pamoate, pectinate, persulfate, 3 phenylproionate, picrate, pivalate, propionate, succinate, sulfate, tartrate, thiocyanate, p toluenesulfonate, and undecanoate. Also, the basic nitrogen-containing groups can be quaternized with such agents as alkyl halides, such as methyl, ethyl, propyl, and butyl chloride, bromides, and iodides; dialkyl sulfates like dimethyl, diethyl, dibutyl, and diamyl sulfates, long chain halides such as decyl, lauryl, myristyl, and stearyl chlorides, bromides and iodides, aralkyl halides like benzyl and phenethyl bromides, and others.

Suitable salts of the compound of formula (I) further include, but are not limited to, cations based on the alkali and alkaline earth metals, such as sodium, lithium, potassium, calcium, magnesium, aluminum salts and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. Other representative organic amines useful for the formation of base addition salts include diethylamine, ethylenediamine, ethanolamine, diethanolamine, piperazine, pyridine, picoline, triethanolamine and the like, and basic amino acids such as arginine, lysine and ornithine.

A preferred compound of formula (I) of the present invention is the PI3K inhibitor 5-(2,6-di-morpholin-4-yl-pyrimidin-4-yl)-4-trifluoromethyl-pyridin-2-ylamine (also known as BKM120) or its hydrochloride salt. The synthesis of this compound is described in WO 2007/084786 as Example 10, the contents of which are incorporated herein by reference.

In another embodiment, other PI3K inhibitors as disclosed in WO2010/029082 can be used. WO2010/029082 describes specific 2-carboxamide cycloamino urea derivatives, which have been found to have highly selective inhibitory activity for the alpha-isoform of phosphatidylinositol 3-kinase (PI3K). A PI3K inhibitor suitable for the present invention is a compound having the following formula (III):

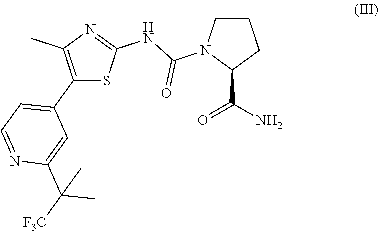 [SMILES: Cc1nc(NC(=O)N2CCC[C@H]2C(N)=O)sc1-c1ccnc(C(C)(C)C(F)(F)F)c1]

(hereinafter “compound of formula (III)” and pharmaceutically acceptable salts thereof. The compound of formula (III) is also known as the chemical compound (S)-Pyrrolidine-1, 2-dicarboxylic acid 2-amide 1-({4-methyl-5-[2-(2,2,2-trifluoro-1,1-dimethyl-ethyl)-pyridin-4-yl]-thiazol-2-yl}-amide). The compound of formula (III), its pharmaceutically acceptable salts and suitable formulations are described in PCT Application No. WO2010/029082, which is hereby incorporated by reference in its entirety, and methods of its preparation have been described, for example, in Example 15 therein. The compound of formula (III) may be present in the form of the free base or any pharmaceutically acceptable salt thereto. Preferably, compound of formula (III) is in the form of its free base.

The PI3K inhibitor of the present invention is selected from the group consisting of 5-(2,6-di-morpholin-4-yl-pyrimidin-4-yl)-4-trifluoromethyl-pyridin-2-ylamine and its hydrochloride salt and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide).

In a preferred embodiment, the PI3K inhibitor of the present invention is the PI3K inhibitor 5-(2,6-di-morpholin-4-yl-pyrimidin-4-yl)-4-trifluoromethyl-pyridin-2-ylamine (also known as BKM120) or its hydrochloride salt.

PI3K Mutations

The present invention includes the method of detecting for or determining the presence of a PIK3CA mutation in a fluid sample such as a blood sample from a patient (e.g., serum or plasma). PIK3CA mutations are known in the art (Mukohara, PI3K mutations in breast cancer: prognostic and therapeutic implications, Breast Cancer: Targets and Therapy, 2015:7 111-123; Particular mutations are disclosed in U.S. Pat. No. 8,026,053). In one embodiment, the method of the present invention can include detecting for or determining the presence of any PIK3CA mutation in exon 1, 2, 5, 7, 9 and/or 20 in the PIK3CA gene. For example, the PIK3CA mutation may comprise one or more of the following mutations R263Q, R277W, R278W, K331E, K333N, K333N, G353D, E1093K, C1258R, E1624K, E1633K, E1634G, Q1636K, H3140K, H3140R, H3140L, and/or H3139Y.

In one example, one or more of the mutations shown in Table 1 can be detected.

TABLE 1

| | | | | | Amino |
|---|---|---|---|---|---|
| | | Nucleotide | Nucleotide | Codon | Acid |
| Gene | Exon | Position | Change | Position | Change |
| PIK3CA | 1 | 263 | G > A | 88 | R > Q |
| PIK3CA | 1 | 277 | C > T | 93 | R > W |
| PIK3CA | 1 | 277 | C > G | 93 | R > W |
| PIK3CA | 1 | 278 | G > A | 93 | R > Q |
| PIK3CA | 1 | 331 | A > G | 111 | K > E |
| PIK3CA | 1 | 333 | G > C | 111 | K > N |
| PIK3CA | 1 | 333 | G > T | 111 | K > N |
| PIK3CA | 2 | 353 | G > A | 118 | G > D |
| PIK3CA | 5 | 1093 | G > A | 365 | E > K |
| PIK3CA | 7 | 1258 | T > C | 420 | C > R |
| PIK3CA | 9 | 1624 | G > A | 542 | E > K |
| PIK3CA | 9 | 1633 | G > A | 545 | E > K |
| PIK3CA | 9 | 1634 | A > G | 545 | E > G |
| PIK3CA | 9 | 1636 | C > A | 546 | Q > K |
| PIK3CA | 20 | 3140 | A > G | 1047 | H > R |
| PIK3CA | 20 | 3140 | A > T | 1047 | H > L |
| PIK3CA | 20 | 3139 | C > T | 1047 | H > Y |

Preparation of Samples

The method of the invention includes detecting a PIK3CA mutation in a bodily fluid which includes a tumor cell such as blood (e.g., serum or plasma) from a patient. As used herein, a “patient” refers to a human or animal, including all mammals such as primates (particularly higher primates. In a preferred embodiment, the patient is a human. Body fluid samples can be obtained from a subject using any of the methods known in the art. Methods for extracting cellular DNA from body fluid samples are also well known in the art. Typically, cells are lysed with detergents. After cell lysis, proteins are removed from DNA using various proteases.

Detection

The amount of ctDNA in a sample is very small so highly sensitive means of measurement is desired to determine the presence of PIK3CA mutation in the ctDNA. The method of the invention can be performed by detecting for the presence of the PI3KCA mutation in ctDNA by polymerase chain reaction (PCR), reverse transcription-polymerase chain reaction (RT-PCR), TaqMan-based assays, direct sequencing, or Beaming.

In one example, the measurement employs amplification on beads in an emulsion using measurement known as BEAMing. BEAMing was named after its components—beads, emulsions, amplification, and magnetics—and essentially converts single DNA template molecules to single beads containing tens of thousands of exact copies of the template (Dressman et al., Proc. Natl. Acad. Sci. USA 2003; 100: 8817-22; U.S. Ser. No. 10/562,840; Diehl et al., NATURE METHODS, VOL. 3 NO. 7, JULY 2006; and Li et al., NATURE METHODS, VOL. 3 NO. 2, FEBRUARY 2006). Specifically, the beaming method includes performing PCR reaction in oil emulsion to immobilize a PCR product derived from one molecule onto one nano particle. The normal and mutated bases are labeled at a site with fluorescent dyes and then detected. Flow cytometry can then be used to quantify the level of mutant PIK3CA DNA present in the plasma or serum (see e.g. Higgins et al. (2012) Clin Cancer Res 18: 3462-3469).

In the method according to the invention any quantitative analysis may be used as far as it can quantitatively determine DNA for each molecule. For example, a wide variety of molecular biology techniques can be used including real-time PCR or next generation sequencers Any type of next generation sequencers may be used as far as it can perform DNA synthesis with DNA polymerase using one DNA molecule as a template and detect fluorescence, emitted light or the like for the reaction of each base in order to determine a base sequence real time, and any base recognition method, lead length, reagent, etc. can also be used for a next generation sequencer.

Administration and Pharmaceutical Composition

In accordance with the present invention, the PI3K inhibitor of the invention may be used for the treatment of a cancer in patients having a PIK3CA mutation in ctDNA. The term “cancer” refers to cancer diseases that can be beneficially treated by the inhibition of PI3K, including, for example, lung and bronchus; prostate; breast; pancreas; colon and rectum; thyroid; liver and intrahepatic bile duct; hepatocellular; gastric; glioma/glioblastoma; endometrial; melanoma; kidney and renal pelvis; urinary bladder; uterine corpus; uterine cervix; ovary; head and neck; multiple myeloma; esophagus; acute myelogenous leukemia; chronic myelogenous leukemia; lymphocytic leukemia; myeloid leukemia; brain; oral cavity and pharynx; larynx; small intestine; non-Hodgkin lymphoma; melanoma; and villous colon adenoma.

In one embodiment, the compound of formula (I) or a pharmaceutically acceptable salt thereof may be used for the treatment of a cancer selected from breast cancer and head and neck cancer.

In a preferred embodiment, the compound of formula (I) or a pharmaceutically acceptable salt thereof may be used for the treatment of a cancer that is breast cancer.

In a further preferred embodiment, the compound of formula (I) or a pharmaceutically acceptable salt thereof may be used for the treatment of a cancer that is breast cancer, wherein the breast cancer is HR+, HER2-negative locally advanced or metastatic breast cancer

The PI3K inhibitor compound of formula (I) or a pharmaceutically acceptable salt thereof is preferably orally administered daily at a dose in the range of from about 0.001 to 1000 mg/kg body weight daily and more preferred from 1.0 to 30 mg/kg body weight. In one embodiment, the dosage compound of formula (I), is in the range of about 10 mg to about 2000 mg per person per day. In one example, 1.0 to 30 mg/kg body weight. In one preferred embodiment, the dosage of compound of formula (I) is in the range of about 60 mg/day to about 120 mg/day, especially if the warm-blooded animal is an adult human. Preferably, the dosage of compound of formula (I) is in the range of about 60 mg/day to about 100 mg/day for an adult human The PI3K inhibitor of the invention may be administered orally to an adult human once daily continuously (each day) or intermittently (e.g, 5 out of 7 days) in a suitable dosage. For example, the phosphatidylinositol 3-kinase inhibitor 5-(2,6-di-morpholin-4-yl-pyrimidin-4-yl)-4-trifluoromethyl-pyridin-2-ylamine or its hydrochloride salt is administered orally to an adult human at a dosage in the range of about 60 mg/day to about 120 mg/day.

In one embodiment, the compound of formula (III) or a pharmaceutically acceptable salt thereof may be used for the treatment of a cancer selected from breast cancer.

In a preferred embodiment, the compound of formula (III) or a pharmaceutically acceptable salt thereof may be used for the treatment of a cancer that is breast cancer.

In a further preferred embodiment, the compound of formula (III) or a pharmaceutically acceptable salt thereof may be used for the treatment of a cancer that is breast cancer, wherein the breast cancer is HR+, HER2-negative locally advanced or metastatic breast cancer

The PI3K inhibitor compound of formula (III) or a pharmaceutically acceptable salt thereof is preferably orally administered at an effective daily dosage of about 1 to 6.5 mg/kg in adults or children. In a 70 kg body weight adult patient, compound of formula (III) or a pharmaceutically acceptable salt thereof is orally administered at a daily dosage of about 70 mg to 455 mg. An effective amount of the therapeutic agent for a particular patient may vary depending on factors such as the condition being treated, the degree of advancement of the disease; the overall health, age, body weight, gender and diet of the patient, the method route and dose of administration and the severity of side effects (see, e.g., Maynard et al., (1996) A Handbook of SOPs for Good Clinical Practice, Interpharm Press, Boca Raton, Fla.; Dent (2001) Good Laboratory and Good Clinical Practice, Urch Publ., London, UK). The optimal effective dosages may be established using routine testing and procedures that are well known in the art.

Data

In performing any of the methods described herein that require determining the presence or absence of a PIK3CA nucleic acid mutation can be used and physicians or genetic counselors or patients or other researchers may be informed of the result. Specifically the result can be cast in a transmittable form of information that can be communicated or transmitted to other researchers or physicians or genetic counselors or patients. Such a form can vary and can be tangible or intangible. The result can be embodied in descriptive statements, diagrams, photographs, charts, images or any other visual forms. For example, images of gel electrophoresis of PCR products can be used in explaining the results. Diagrams showing a variant is present or absent are also useful in indicating the testing results. These statements and visual forms can be recorded on a tangible media such as papers, computer readable media such as floppy disks, compact disks, etc., or on an intangible media, e.g., an electronic media in the form of email or website on internet or intranet. In addition, the result can also be recorded in a sound form and transmitted through any suitable media, e.g., analog or digital cable lines, fiber optic cables, etc., via telephone, facsimile, wireless mobile phone, internet phone and the like. All such forms (tangible and intangible) would constitute a “transmittable form of information”. Thus, the information and data on a test result can be produced anywhere in the world and transmitted to a different location. For example, when a genotyping assay is conducted offshore, the information and data on a test result may be generated and cast in a transmittable form as described above. The test result in a transmittable form thus can be imported into the U.S. Accordingly, the present disclosure also encompasses a method for producing a transmittable form of information containing data on whether a mutation occurs in an individual. This form of information is useful for predicting the responsiveness of a patient to treatment with at PI3K inhibitor, for selecting a course of treatment based upon that information, and for selectively treating a patient based upon that information.

Kits

The invention further provides kits for determining whether a mutation exists at a particular position of the PIK3CA gene as shown in Table 1. In a preferred embodiment, the kits are useful for selecting patients who will specifically benefit from treatment with a PI3K inhibitor 5-(2,6-di-morpholin-4-yl-pyrimidin-4-yl)-4-trifluoromethyl-pyridin-2-ylamine or its hydrochloride salt. A kit can comprise primers and/probes useful for detecting a mutation of the PIK3CA gene. A kit may further comprise nucleic acid controls, buffers, and instructions for use.

In an alternative embodiment, the kits are useful for selecting patients who will specifically benefit from treatment with a PI3K inhibitor compound (S)-Pyrrolidine-1, 2-dicarboxylic acid 2-amide 1-({4-methyl-5-[2-(2,2,2-trifluoro-1,1-dimethyl-ethyl)-pyridin-4-yl]-thiazol-2-yl})-amide) or a pharmaceutically acceptable salt thereof.

Other features, objects, and advantages of the invention will be apparent from the description and drawings, and from the claims and the Enumerated Embodiments below. Specifically, the present disclosure provides the following aspects, advantageous features and specific 1 embodiments, respectively alone or in combination, as listed in the following Enumerated

### EMBODIMENTS

1. A method of treating a patient having a cancer, comprising administering a therapeutically effective amount of 5-(2,6-di-morpholin-4-yl-pyrimidin-4-yl)-4-trifluoromethyl-pyridin-2-ylamine or its hydrochloride salt to the patient on the basis of the patient having been determined to have in their circulating tumor DNA (ctDNA) a PIK3CA mutation.

2. A method of treating a patient having a cancer, comprising either:
administering a therapeutically effective amount of 5-(2,6-di-morpholin-4-yl-pyrimidin-4-yl)-4-trifluoromethyl-pyridin-2-ylamine or its hydrochloride salt to the patient on the basis of the patient having been determined to have in their ctDNA a PIK3CA mutation; or
administering a therapeutically effective amount of a therapeutic other than 5-(2,6-di-morpholin-4-yl-pyrimidin-4-yl)-4-trifluoromethyl-pyridin-2-ylamine or its hydrochloride salt to the patient on the basis of the patient not having been determined to have in their ctDNA a PIK3CA mutation.

3. The method according to any of the above Enumerated Embodiments, wherein the therapeutic other than 5-(2,6-di-morpholin-4-yl-pyrimidin-4-yl)-4-trifluoromethyl-pyridin-2-ylamine or its hydrochloride salt is selected from the group consisting of fulvestrant, trastuzumab, lapatinib, gefinitib, erlotinib, paclitaxel, everolimus, methotrexate, fluorouracil, anastrozole, exemestane, capecitabine, cyclophosphamide, letrozole, toremifene, gemcitabine hydrochloride, goserelin acetate, palbociclib, megestrol acetate, tamoxifen, palbociclib, pertuzumab, or vinblastine and combinations thereof.

4. The method according to any of the above Enumerated Embodiments, wherein the cancer is selected from a cancer of the lung and bronchus; prostate; breast; pancreas; colon and rectum; thyroid; liver and intrahepatic bile duct; hepatocellular; gastric; glioma/glioblastoma; endometrial; melanoma; kidney and renal pelvis; urinary bladder; uterine corpus; uterine cervix; ovary; head and neck; multiple myeloma; esophagus; acute myelogenous leukemia; chronic myelogenous leukemia; lymphocytic leukemia; myeloid leukemia; brain; oral cavity and pharynx; larynx; small intestine; non-Hodgkin lymphoma; melanoma; and villous colon adenoma.

5. The method according to any of the above Enumerated Embodiments, wherein the cancer is selected from breast cancer and head and neck cancer.

6. The method according to any of the above Enumerated Embodiments, wherein the cancer is breast cancer.

7. A method of treating a patient having a cancer with a PI3K inhibitor, comprising: selecting the patient for treatment with 5-(2,6-di-morpholin-4-yl-pyrimidin-4-yl)-4-trifluoromethyl-pyridin-2-ylamine or its hydrochloride salt on the basis of the patient having been determined to have in their circulating tumor DNA (ctDNA) a PIK3CA mutation; and thereafter, administering a therapeutically effective amount of 5-(2,6-di-morpholin-4-yl-pyrimidin-4-yl)-4-trifluoromethyl-pyridin-2-ylamine or its hydrochloride salt to the patient.

8. A method of treating a patient having a cancer with a PI3K inhibitor, comprising:
a) assaying a blood or a plasma sample comprising ctDNA from the patient having breast cancer for the presence of a PIK3CA mutation in the ctDNA; and
b) administering a therapeutically effective amount of 5-(2,6-di-morpholin-4-yl-pyrimidin-4-yl)-4-trifluoromethyl-pyridin-2-ylamine or its hydrochloride salt to the patient on the basis of that patient having been determined to have a PIK3 CA mutation.

9. The method of any of the above Enumerated Embodiments, wherein the PIK3CA mutation includes a mutation in exon 1, 2, 5, 7, 9 and/or 20 in the PIK3CA gene.

10. The method of Enumerated Embodiment 9, wherein the PIK3CA mutation comprises one or more of the following mutations R263Q, R277W, R278W, K331E, K333N, K333N, G353D, E1093K, C1258R, E1624K, E1633K, E1634G, Q1636K, H3140K, H3140R, H3140L, and/or H3139Y.

11. The method of any of the above Enumerated Embodiments, wherein the presence of the PI3KCA mutation in ctDNA is detected by a technique selected from the group consisting of polymerase chain reaction (PCR), reverse transcription-polymerase chain reaction (RT-PCR), TaqMan-based assays, direct sequencing, or Beaming

12. The method according to Enumerated Embodiment 8, wherein the step of administering comprises administering orally about 60 mg to about 120 mg per said patient.

13. 5-(2,6-di-morpholin-4-yl-pyrimidin-4-yl)-4-trifluoromethyl-pyridin-2-ylamine or its hydrochloride salt for use in treating a cancer, characterized in that a therapeutically effective amount of 5-(2,6-di-morpholin-4-yl-pyrimidin-4-yl)-4-trifluoromethyl-pyridin-2-ylamine or its hydrochloride salt is administered to the patient on the basis of said patient having been determined to comprise in their circulating tumor DNA (ctDNA) a PIK3CA mutation.

14. The 5-(2,6-di-morpholin-4-yl-pyrimidin-4-yl)-4-trifluoromethyl-pyridin-2-ylamine or its hydrochloride salt according to Enumerated Embodiment 10, characterized in that a therapeutically effective amount of the 5-(2,6-di-morpholin-4-yl-pyrimidin-4-yl)-4-trifluoromethyl-pyridin-2-ylamine or its hydrochloride salt is administered to the patient on the basis of said patient having one or more mutations R263Q, R277W, R278W, K331E, K333N, K333N, G353D, E1093K, C1258R, E1624K, E1633K, E1634G, Q1636K, H3140K, H3140R, H3140L, and/or H3139Y in the PIK3CA gene.

15. A method of predicting the likelihood that a patient having a cancer will respond to treatment with 5-(2,6-di-morpholin-4-yl-pyrimidin-4-yl)-4-trifluoromethyl-pyridin-2-ylamine or its hydrochloride salt, comprising assaying a blood or serum sample comprising a tumor cell obtained from the patient for the presence of a PIK3 CA mutation, wherein:
a) the presence of the PIK3CA mutation is indicative of an increased likelihood that the patient will respond to treatment with 5-(2,6-di-morpholin-4-yl-pyrimidin-4-yl)-4-trifluoromethyl-pyridin-2-ylamine or its hydrochloride salt; and
b) the absence of the PIK3CA mutation is indicative of a decreased likelihood that the patient will respond to treatment with 5-(2,6-di-morpholin-4-yl-pyrimidin-4-yl)-4-trifluoromethyl-pyridin-2-ylamine or its hydrochloride salt.

16. The method of Enumerated Embodiment 15, wherein the tumor cell is a circulating tumor cell.

17. The method of Enumerated Embodiment 16, wherein the sample comprises circulating tumor DNA (ctDNA).

18. The method according to any one of Enumerated Embodiments 7 to 17, wherein the cancer is selected from a cancer of the lung and bronchus; prostate; breast; pancreas; colon and rectum; thyroid; liver and intrahepatic bile duct; hepatocellular; gastric; glioma/glioblastoma; endometrial; melanoma; kidney and renal pelvis; urinary bladder; uterine corpus; uterine cervix; ovary; head and neck; multiple myeloma; esophagus; acute myelogenous leukemia; chronic myelogenous leukemia; lymphocytic leukemia; myeloid leukemia; brain; oral cavity and pharynx; larynx; small intestine; non-Hodgkin lymphoma; melanoma; and villous colon adenoma.

19. The method according to any one of Enumerated Embodiments 7 to 17, wherein the cancer is selected from breast cancer and head and neck cancer.

20. The method according to any one of Enumerated Embodiments 7 to 17, wherein the cancer is breast cancer.

21. The method according to any one of the preceding Enumerated Embodiments, wherein the breast cancer is HR+, HER2-negative locally advanced or metastatic breast cancer.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

### Example 1

Study CBKM120F2302 was a multicenter, randomized, double-blind, placebo-controlled Phase-III trial designed to determine the efficacy and safety of treatment with buparlisib plus fulvestrant vs. placebo plus fulvestrant in postmenopausal women with HR+, HER2-negative locally advanced or metastatic breast cancer whose disease had progressed on or after AI therapy.

For the Study, patients were selected according to the following inclusions and exclusion criteria: Inclusion Criteria:
Locally advanced or metastatic breast cancer
HER2-negative and hormone receptor-positive status (common breast cancer classification tests)
Postmenopausal woman
A tumor sample must be shipped to a Novartis designated laboratory for identification of biomarkers (PI3K activation status)
Progression or recurrence of breast cancer while on or after aromatase inhibitor treatment
Measurable disease or non measurable disease bone lesions in the absence of measurable disease as per Responce Evaluation Criteria in Solid Tumors 1.1
Adequate bone marrow and organ function defined by laboratory values

Exclusion Criteria:
Previous treatment with PI3K inhibitors, AKT inhibitors, mTOR inhibitor or fulvestrant
More than one prior chemotherapy line for metastatic disease
Symptomatic brain metastases
Increasing or chronic treatment (>5 days) with corticosteroids or another immunosuppressive agent
Active heart (cardiac) disease as defined in the protocol
Anxiety (Common Terminology Criteria for Adverse Events Grade ≥3) or history/evidence of depression or other mood disorders
GAD-7 (7-item Generalized Anxiety Disorder) mood scale score ≥15, PHQ-9 (9-item Patient Health Questionaire) score >12, or positive response to PHQ-9 question 9 relating to suicidal ideation.

Approximately 1200 patients were to be randomized in a 1:1 ratio. Enrollment was to continue until a minimum of 842 patients were randomized in the main cohort, including ≥334 patients with activated PI3K pathway status. Randomized patients were included in one of two cohorts:
Main cohort: consisting of patients with known PI3K pathway activation status (activated or non-activated)
PI3K unknown cohort: comprising patients with unknown PI3K pathway status

Per Amendment 2 to the protocol, mandatory blood collection at study entry was implemented in June 2013 as part of Amendment 2 to the protocol. Testing of ctDNA was designed to assess the presence of PIK3CA hot-spot mutations in exons 1, 5, 7, 9, and 20 using Beads, Emulsification, Amplification, and Magnetics (BEAMing) technology. In addition, a prespecified exploratory PFS analysis based on the PIK3CA mutation status by ctDNA was detailed in the statistical analysis plan.

Per Amendment 3 to the protocol, the Full population was defined as comprising both the main and PI3K unknown cohorts, and was representative of the overall HR+, HER2-negative breast cancer population.

After a 14-day run-in treatment phase consisting of fulvestrant 500 mg administered alone on Cycle 1 Day 1, patients were randomized (1:1) on Cycle 1 Day 15 to one of two treatment arms: buparlisib plus fulvestrant or placebo plus fulvestrant. Randomization was stratified according to PI3K pathway activation status (activated, non-activated, or unknown) and visceral disease status (present or absent). Absence of visceral disease was defined as having lesions only in bone and/or skin, and/or nodes, and/or breast, and/or soft tissues; the presence of visceral disease was defined as lesions in any other location.

The primary objectives of the trial were to determine whether treatment with buparlisib plus fulvestrant prolonged progression-free survival (PFS) per local radiology review relative to placebo plus fulvestrant in the following populations:
Full population: all randomized patients irrespective of the PI3K pathway activation status (i.e. activated, non-activated, or unknown)
Main cohort: all randomized patients with known PI3K pathway activation status (either activated or non-activated)
Activated PI3K pathway subpopulation: all randomized patients with an activated PI3K pathway status

The PI3K pathway activation status was defined based on analysis of archival tumor samples as:
A mutation in the PIK3CA gene in one or more of exons 1, 7, 9, or 20 as assessed by Sanger sequencing, and/or
Loss of phosphotensin homolog (PTEN) expression (<10% of tumor cells expressing PTEN at 1+ level by immunohistochemistry [IHC] and no tumor cells staining with an intensity >1+)

Enrollment to the study commenced in September 2012 and completed in July 2014. A total of 1147 patients were randomly assigned (1:1) to receive treatment with either buparlisib (100 mg daily) plus fulvestrant (500 mg) (n=576) or placebo plus fulvestrant (500 mg) (n=571). There were 851 patients randomized in the Main cohort [buparlisib plus fulvestrant: n=427; placebo plus fulvestrant: n=424] [Activated: n=372 (43.7%) and Non-activated: n=479 (56.2%)]. The cut-off date for this primary analysis was 29 Apr. 2015.

Tumor assessments were performed 6 weeks after the date of randomization and subsequently every 8 weeks until disease progression. Imaging data used for tumor assessments during the treatment and follow-up phases were collected centrally and prospectively reviewed by a blinded independent review committee.

All patients were followed for survival status every 3 months irrespective of their reason for treatment discontinuation (except if consent was withdrawn, the patient refused survival follow-up, or the patient was lost to follow-up). Additional survival assessments outside the 3-month follow-up schedule were permitted if a survival update was required to meet safety or regulatory needs.

An Independent Data Monitoring Committee (IDMC) was responsible for monitoring the safety, buparlisib PK, and efficacy (assessing criteria for early stopping due to futility based on PFS) of the study participants, ensuring that the trial was being conducted with the highest scientific and ethical standards, and making appropriate recommendations based on the reported data.

A Study Steering Committee (SSC) was established to ensure the transparent management of the trial according to the protocol.

The final PFS analysis was performed in June 2015 after the prespecified number of events was reached (corresponding to a 29 Apr. 2015 data cut-off).

Results in the Full Population

In the Full Population, the main findings are the following:
Baseline characteristics of the Full population were generally well balanced between the two treatment arms and consistent with a patient population with advanced HR+ breast cancer after failure of prior therapies, including an AI
Patient Disposition: Progression of disease was the most common reason for treatment discontinuation (54.3% of the patients in the buparlisib plus fulvestrant arm and 73% in the placebo plus fulvestrant arm). Adverse event (AE) was reported as primary reason for treatment discontinuation in 13.2% patients in buparlisib plus fulvestrant arm vs. 1.8% patients in placebo plus fulvestrant arm (Table 1-1 Patient disposition (Full analysis set—Full population)):

| | Buparlisib plus | | Placebo plus | | | |
|---|---|---|---|---|---|---|
| | fulvestrant | | fulvestrant | | All patients | |
| | N = 576 | | N = 571 | | N = 1147 | |
| Disposition reason | n (%) | | n (%) | | n (%) | |
| Patients randomized | | | | | | |
| Untreated | 2 | (0.3) | 2 | (0.4) | 4 | (0.3) |
| Treated | 574 | (99.7) | 569 | (99.6) | 1143 | (99.7) |
| Patients treated | | | | | | |
| Treatment phase | 93 | (16.1) | 94 | (16.5) | 187 | (16.3) |
| ongoing ¹ | | | | | | |
| End of treatment | 481 | (83.5) | 475 | (83.2) | 956 | (83.3) |
| Reason for not | | | | | | |
| being treated | | | | | | |
| Physician decision | 1 | (0.2) | 1 | (0.2) | 2 | (0.2) |
| Adverse event | 1 | (0.2) | 0 | | 1 | (0.1) |
| Death | 0 | | 1 | (0.2) | 1 | (0.1) |
| Primary reason for | | | | | | |
| end of treatment | | | | | | |
| Progressive disease | 313 | (54.3) | 417 | (73.0) | 730 | (63.6) |
| Adverse event(s) | 76 | (13.2) | 10 | (1.8) | 86 | (7.5) |
| Subject/guardian | 51 | (8.9) | 18 | (3.2) | 69 | (6.0) |
| decision | | | | | | |
| Physician decision | 23 | (4.0) | 21 | (3.7) | 44 | (3.8) |
| Death | 7 | (1.2) | 5 | (0.9) | 12 | (1.0) |
| Non-compliance with | 8 | (1.4) | 1 | (0.2) | 9 | (0.8) |
| study treatment | | | | | | |
| Protocol deviation | 2 | (0.3) | 3 | (0.5) | 5 | (0.4) |

| | | | | | |
|---|---|---|---|---|---|
| Lost to follow-up | 1 | (0.2) | 0 | 1 | (0.1) |

¹ Patients ongoing at the time of the 29 Apr. 2015 data cut-off

The study met its primary objectives for PFS in both the Full population and Main cohort, and that there was a trend in favor of the buparlisib plus fulvestrant arm for prolonged PFS in the activated PI3K pathway subpopulation based on archival tumor tissue although this did not reach statistical significance (Table 1-2).

TABLE 1-2
Progression-free survival per local imaging review (FAS)

| | | | | | Activated PI3K | |
|---|---|---|---|---|---|---|
| | Full population | | Main cohort | | pathway | |
| | Buparlisib | Placebo | Buparlisib | Placebo | Buparlisib | Placebo |
| | plus | plus | plus | plus | plus | plus |
| | fulvestrant | fulvestrant | fulvestrant | fulvestrant | fulvestrant | fulvestrant |
| | N = 576 | N = 571 | N = 427 | N = 424 | N = 188 | N = 184 |
| No. of PFS events - n | 349 (60.6) | 435 (76.2) | 271 (63.5) | 324 (76.4) | 116 (61.7) | 144 (78.3) |
| (%) | | | | | | |
| No. censored - n (%) | 227 (39.4) | 136 (23.8) | 156 (36.5) | 100 (23.6) | 72 (38.3) | 40 (21.7) |
| Median PFS (mo) | 6.9 | 5.0 | 6.8 | 4.5 | 6.8 | 4.0 |
| 95% CI | 6.8, 7.8 | 4.0, 5.2 | 5.0, 7.0 | 3.3, 5.0 | 4.9, 7.1 | 3.1, 5.2 |
| Improvement in | 1.9 | | 2.3 | | 2.8 | |
| median PFS (mo) | | | | | | |
| Hazard ratio | 0.78 | | 0.80 | | 0.76 | |
| (stratified Cox model) | | | | | | |
| 95% CI | 0.67, 0.89 | | 0.68, 0.94 | | 0.60, 0.97 | |
| One-sided p-value¹ | <0.001 | | 0.003 | | 0.014 | |
| (stratified log-rank | | | | | | |
| test) | | | | | | |

CI—Confidence interval;
mo—Months;
PFS—Progression-free survival
¹As governed by the gatekeeping procedure controlling an overall 2.5% type-1 error, PFS in the Main cohort was tested at the one-sided 2% level of significance. PFS in the PI3K pathway activated subpopulation was tested at the one-sided 1% level of significance as PFS in the Main cohort was statistically significant at the one-sided 2% level of significance. PFS in the Full population was tested at the one-sided 1.4% level of significance as PFS in the Main cohort was statistically significant at the one-sided 2% level of significance. Both the log-rank test and Cox model were stratified by PI3K pathway activation status and visceral disease status. Within the activated PI3K pathway status, the stratified log-rank test and Cox regression model were stratified by visceral disease status.

The PFS increase in the activated PI3K pathway subpopulation was not statistically significant based on the one sided p value. PI3K pathway activation was assessed in archival tumor tissue provided at screening, defined as PIK3CA mutation by Sanger sequencing (specified mutations in exons 1, 7, 9 or 20) and/or loss of PTEN expression by immunohistochemistry (<1+ expression in <10% of cells). FIG. 1 shows the probability of PFS survival (%) for the buparlisib plus fulvestrant arm relative to the placebo plus fulvestrant arm for the PI3K Activated Group (Archival Tissue).

Consistent improvements in median PFS of approximately 2 months were observed in the buparlisib plus fulvestrant arm relative to the placebo plus fulvestrant arm for both the Full population and the Main cohort. An improvement of 2.8 months was observed in the activated PI3K pathway subpopulation. Improvements in PFS were consistent between local and independent central imaging reviews.

Overall response rate (ORR) and clinical benefit rate (CBR) were also both suggestive of improvements in favor of buparlisib plus fulvestrant (Table 1-3).

TABLE 1-3
Objective response rates and clinical benefit
rates (Full analysis set- Full population)

| | Buparlisib plus | | Placebo plus | |
|---|---|---|---|---|
| | fulvestrant | | fulvestrant | |
| | N = 576 | | N = 571 | |
| | (%) | 95% CI | n (%) | 95% CI |
| Objective response | 11.8 | (9.3, 14.7) | 7.7 | (5.7, 10.2) |
| rate (ORR: CR + PR) | | | | |
| Median duration of | 7.4 | | 7.5 | |
| response (months) | | | | |
| Clinical benefit rate | 43.8 | (39.7, 47.9) | 42.0 | (37.9, 46.2) |
| (CR + PR and SD + | | | | |
| Non-CR/Non-PD >24 | | | | |
| weeks) | | | | |

Overall safety and tolerability profile of buparlisib was consistent with prior experience in single-arm and combination studies and with the class effects of PI3K inhibitors; adverse events (AEs) reported were generally manageable (based on the guidance provided in the protocol).

Results in the PIK3CA ctDNA Population

Clinically relevant treatment effect was observed in a prospectively defined analysis based on circulating tumor DNA (ctDNA). Circulating tumor DNA was successfully collected and analyzed in 587 of the 1147 patients (51.2%) randomized to treatment (Table 1-4). All 587 plasma samples collected had a matching archival tumor tissue samples. The ctDNA analysis was pre-planned, and data were generated prior to the study database lock. The samples were collected appropriately and prepared for shipping and storage for the specific purpose of extracting ctDNA and analyzing for 15 hotspot PIK3CA mutations covering functional hotspots in the exon 1, 7, 9 and 20 using BEAMing technology, which provided the ability to detect an additional 18.5% samples with PIK3CA mutation.

Of these 587 patients, 200 were PIK3CA^{mut} by ctDNA and 387 were PIK3CA^{wt} by ctDNA. Of the 200 patients with PIK3CA^{wt} by ctDNA, 87 (43.5%) received treatment with buparlisib plus fulvestrant and 113 (56.5%) placebo plus fulvestrant therapy. Of the 387 patients with PIK3CA^{WT} by ctDNA, 199 (51.4%) received treatment with buparlisib plus fulvestrant and 188 (48.6%) placebo plus fulvestrant. As of the 29 Apr. 2015 data cut-off, approximately 20% of the patients with available ctDNA data were ongoing in the study.

TABLE 1-4
Analysis sets

| | Buparlisib | | |
|---|---|---|---|
| | plus | Placebo plus | |
| | fulvestrant | fulvestrant | All patients |
| | N = 576 | N = 571 | N = 1147 |
| Analysis set | n (%) | n (%) | n (%) |
| Full analysis set | 576 (100.0) | 571 (100.0) | 1147 (100.0) |
| Patients without ctDNA | 290 (50.3) | 270 (47.3) | 560 (48.8) |
| Patients with ctDNA | 286 (49.7) | 301 (52.7) | 587 (51.2) |
| ctDNA mutant (PIK3CA^{mut}) | 87 (15.1) | 113 (19.8) | 200 (17.4) |
| ctDNA wild type | 199 (34.5) | 188 (32.9) | 387 (33.7) |
| (PIK3CA^{WT}) | | | |
| Safety set | 573 (99.5) | 570 (99.8) | 1143 (99.7) |
| Patients without ctDNA | 288 (50.3) | 269 (47.2) | 557 (48.7) |
| Patients with ctDNA | 285 (49.7) | 301 (52.8) | 586 (51.3) |
| ctDNA mutant (PIK3CA^{mut}) | 87 (15.2) | 112 (19.6) | 199 (17.4) |
| ctDNA wild type | 198 (34.6) | 189 (33.2) | 387 (33.9) |
| (PIK3CA^{WT}) | | | |

ctDNA—Circulating tumor DNA

Baseline demography and disease characteristics in the ctDNA subpopulations were consistent with the Full population and reflected a patient population with HR+, HER2-negative breast cancer refractory to AI therapy.

Patient Disposition:

Approximately 20% of the patients with available ctDNA data were ongoing in the study and a greater proportion of patients continued to receive therapy with the buparlisib treatment regimen in the PIK3CA^{mut} population at the time of data cut-off. In the PIK3CA^{mut} population progression of disease was the most common reason for treatment discontinuation (49.4% of the patients in the buparlisib plus fulvestrant arm and 73.5% in the placebo plus fulvestrant arm) (Table 1-5).

TABLE 1-5
Patient disposition in patients with ctDNA

| | PIK3CA^{mut} by ctDNA | | PIK3CA^{WT} by ctDNA | |
|---|---|---|---|---|
| | N = 200 | | N = 387 | |
| | Buparlisib | | Buparlisib | |
| | plus | Placebo plus | plus | Placebo plus |
| | fulvestrant | fulvestrant | fulvestrant | fulvestrant |
| | N = 87 | N = 113 | N = 199 | N = 188 |
| | n (%) | n (%) | n (%) | n (%) |
| Patients randomized | | | | |
| Untreated | 0 | 1 (0.9) | 0 | 0 |
| Treated | 87 (100.0) | 112 (99.1 ) | 199 (100.0) | 188 (100.0) |
| Patients treated | | | | |
| Treatment phase ongoing | 17 (19.5) | 13 (11.5) | 37 (18.6) | 51 (27.1) |
| End of treatment | 70 (80.5) | 99 (87.6) | 162 (81.4) | 137 (72.9) |
| Primary reason for end of | | | | |
| treatment | | | | |
| Progressive disease | 43 (49.4) | 83 (73.5) | 107 (53.8) | 122 (64.9) |
| Adverse event | 9 (10.3) | 3 (2.7) | 26 (13.1) | 1 (0.5) |
| Subject/guardian decision | 8 (9.2) | 3 (2.7) | 13 (6.5) | 6 (3.2) |
| Physician decision | 6 (6.9) | 4 (3.5) | 11 (5.5) | 7 (3.7) |
| Death | 1 (1.1) | 3 (2.7) | 3 (1.5) | 1 (0.5) |
| Non-compliance with study | 2 (2.3) | 1 (0.9) | 1 (0.5) | 0 |
| treatment | | | | |
| Protocol deviation | 1 (1.1) | 2 (1.8) | 0 | 0 |
| Lost to follow-up | 0 | 0 | 1 (0.5) | 0 |

ctDNA—Circulating tumor DNA

Efficacy analysis in the PIK3CA^{mut} by ctDNA subpopulation showed:
A clinically meaningful 44% reduction in the risk of progression or death in the buparlisib plus fulvestrant treatment arm (HR 0.56; 95% CI: 0.39, 0.80), and a 3.8-month prolongation in median PFS from 3.2 to 7.0 months compared with the placebo plus fulvestrant arm (Table 1-6). No such PFS benefit was noted in the PIK3CA^{WT} by ctDNA subpopulation (HR 1.05; 95% CI: 0.82, 1.34), with median PFS for both treatment arms of 6.8 months.

TABLE 1-6
Progression-free survival analysis in patients
with ctDNA per local imaging review (FAS)

| | PIK3CA^{mut} by ctDNA | | PIK3CA^{WT} by ctDNA | |
|---|---|---|---|---|
| | Buparlisib | Placebo | Buparlisib | Placebo |
| | plus | plus | plus | plus |
| | fulvestrant | fulvestrant | fulvestrant | fulvestrant |
| | N = 87 | N = 113 | N = 199 | N = 188 |
| Median PFS (mo) | 7.0 | 3.2 | 6.8 | 6.8 |
| 95% CI | 5.0, 10.0 | 2.0, 5.1 | 4.7, 8.5 | 4.7, 8.6 |
| Improvement in | 3.8 | | 0 | |
| median PFS (mo) | | | | |
| Hazard ratio | 0.56 | | 1.05 | |
| (unstratified) | | | | |
| 95% CI | 0.39, 0.80 | | 0.82, 1.34 | |

CI—Confidence interval;
ctDNA—Circulating tumor DNA;
mo—Months;
PFS—Progression-free survival

This is depicted in FIG. 2.
Discordance was noted for the 200 samples deemed to be PIK3CA^{mut} by ctDNA where, by Sanger sequencing, 99 were mutant, 64 were wildtype, and 36 were unknown for PIK3CA status in the archival tissue. The PFS benefit is maintained in all the 3 Sanger subgroups for the PIK3CA^{mut} by ctDNA subpopulations, irrespective of the Sanger Sequencing mutation status. In the 64 patients who had Sanger PIK3CA wildtype, there was a clinically meaningful improvement of ˜3 months with a median PFS of 4.6 months vs. 1.5 months (HR=0.58) in favor of buparlisib arm (Table 1-7).

TABLE 1-7
Progression-free survival in ctDNA PIK3CA mutant and WT subgroups per local imaging review and by PIK3CA
mutation status by Sanger sequencing

| | | Event/N (%) | | Median PFS (mo) (95% CI) | | |
|---|---|---|---|---|---|---|
| | | Buparlisib plus | Placebo plus | Buparlisib plus | Placebo plus | Unstratified |
| | N (%) | fulvestrant | fulvestrant | fulvestrant | fulvestrant | HR (95% CI) |
| PIK3CA^{mut} | 200 (17.4) | 48/87 (55.2) | 90/113 (79.6) | 7.0 (5.0, 10.0) | 3.2 (2.0, 5.1) | 0.56 (0.39, 0.80) |
| Sanger mutated | 99 (8.6) | 23/42 (54.8) | 46/57 (80.7) | 7.1 (4.6, 10.0) | 3.4 (2.0, 5.3) | 0.58 (0.35, 0.96) |
| Sanger wild type | 64 (5.6) | 17/27 (63.0) | 29/37 (78.4) | 4.6 (3.3, 15.1) | 1.5 (1.4, 5.1) | 0.58 (0.32, 1.05) |
| Sanger unknown | 36 (3.1) | 7/17 (41.2) | 15/19 (78.9) | 7.0 (5.0, NE) | 5.1 (1.4, 14.2) | 0.44 (0.18, 1.10) |
| PIK3CA^{wt} | 387 (33.7) | 124/199 (62.3) | 126/188 (67.0) | 6.8 (4.7, 8.5) | 6.8 (4.7, 8.6) | 1.05 (0.82, 1.34) |
| Sanger mutated | 40 (3.5) | 10/21 (47.6) | 10/19 (52.6) | 4.4 (1.6, NE) | 10.7 (3.0, NE) | 1.18 (0.49, 2.85) |
| Sanger wild type | 243 (21.2) | 82/123 (66.7) | 84/120 (70.0) | 5.1 (3.5, 8.5) | 4.7 (3.3, 8.5) | 0.98 (0.72, 1.32) |
| Sanger unknown | 100 (8.7) | 31/54 (57.4) | 29/46 (63.0) | 8.5 (5.7, 8.9) | 6.9 (5.1, 14.2) | 1.15 (0.69, 1.91) |

CI—Confidence interval;
ctDNA—Circulating tumor DNA;
HR—Hazard ratio;
mo—Months;
NE—Not estimable;
PFS—Progression-free survival

Overall response rate and clinical benefit rate: The ORR for the buparlisib plus fulvestrant treatment arm was 18.4% compared with 3.5% for the placebo plus fulvestrant arm and the respective CBRs were 47.1% vs. 31.9%. Median duration of response was 7.5 months vs. 4.5 months for buparlisib vs. control arm in the PIK3CA^{mut} by ctDNA subpopulation (Table 1-8).

TABLE 1-8
Objective response rates and clinical
benefit rates in ctDNA subpopulations

| | PIK3CA^{mut} by ctDNA | | PIK3CA^{WT} by ctDNA | |
|---|---|---|---|---|
| | Buparlisib | Placebo | Buparlisib | Placebo |
| | plus | plus | plus | plus |
| | fulvestrant | fulvestrant | fulvestrant | fulvestrant |
| | N = 87 | N = 113 | N = 199 | N = 188 |
| Objective response | 18.4 | 3.5 | 11.6 | 10.6 |
| rate (%) | | | | |
| 95% CI | 10.9, 28.1 | 1.0, 8.8 | 7.5, 16.8 | 6.6, 16.0 |
| Median duration | 7.5 | 4.5 | 7.4 | 11.1 |
| of response for | | | | |
| responders (months) | | | | |
| Clinical benefit | 47.1 | 31.9 | 42.7 | 50.0 |
| rate ¹ (%) | | | | |
| 95% CI | 36.3, 58.1 | 23.4, 41.3 | 35.7, 49.9 | 42.6, 57.4 |

¹ Clinical benefit rate = best response of complete response, partial response, or stable disease for ≥24 weeks
CI—Confidence interval;
ctDNA—Circulating tumor DNA

Waterfall plots based on PIK3CA^{mut} by ctDNA status showed that more patients treated with buparlisib plus fulvestrant experienced tumor shrinkage compared with those receiving placebo plus fulvestrant (FIG. 3)
A trend in favor of the buparlisib plus fulvestrant arm in OS for the PIK3CA^{mut} subpopulation (HR 0.62; 95% CI: 0.36, 1.05) (FIG. 4), although these data are currently immature (with 21 and 37 deaths reported as of the data cut-off date for the buparlisib plus fulvestrant and placebo plus fulvestrant arms, respectively).

Efficacy analysis in the PIK3CA WT by ctDNA subpopulation showed:
No PFS benefit for patients categorized as PIK3CA^{WT} by ctDNA (median PFS for both arms was 6.8 months) (HR 1.05; 95% CI: 0.82, 1.34) (Table 1-6)
The 3.8-month prolongation of median PFS was not observed when PFS was analyzed based on the 276 patients with PIK3CA mutations as determined by Sanger sequencing in archival tumor tissue using Sanger sequencing; median PFS was 5.3 months for the buparlisib plus fulvestrant arm vs. 4.7 months for the placebo plus fulvestrant arm (HR 0.81; 95% CI: 0.60, 1.08)
No difference in OS is currently observed between the two treatment arms for the PIK3CA^{WT} by ctDNA subpopulation (FIG. 4).
Discordance was observed between PIK3CA mutation status assessments by ctDNA vs. Sanger sequencing in the tumor tissue. As shown in Table 1-7, of the 200 samples with PIK3CA^{mut} by ctDNA, 99 had mutation(s), 64 were wild-type for PIK3CA, and 36 were deemed to be of unknown status for PIK3CA in the archival tumor tissue. Discordance was also noted for the 387 samples deemed to be PIK3CA^{WT} by ctDNA where, by Sanger sequencing, 243 were wild-type, 40 were mutant, and 100 were unknown for PIK3CA status in the archival tumor tissue
The PFS benefit was maintained in the PIK3CA^{mut} by ctDNA subgroups, irrespective of the Sanger sequencing mutation status (Table 1-7)

The following table 1-9 provides a comparison of the efficacy of the treatment regimens based on PIK3CA mutation status in archival tumor tissue in the Study.

TABLE 1-9
Efficacy of Study regimens based on PIK3CA mutation status
in archival tumor tissue and baseline ctDNA samples

| | | | | Data based on Plasma Samples | |
|---|---|---|---|---|---|
| | | Data based on Archival Tumor Samples | | analyzed for PIK3CA in ctDNA by | |
| | | analyzed by Sanger Sequencing) | | BEAMing assay) | |
| | | mPFS (95% | | mPFS (95% | |
| PIK3CA | | CI) months | HR | CI) months | HR |
| Status | Treatment | events n/N | (95% CI) | events n/N | (95% CI) |
| PIK3CA | Placebo | 4.7 (3.2, 6.3) | 0.80 | 3.2 (2-5.1) | 0.56 |
| Mutated | | n/N = 106/140 | (0.6-1.08) | n/N = 90/113 | (0.39-0.80) |
| | Investigational | 5.3 (4.6, 7.1) | | 7 (5-10) | |
| | Arm | n/N = 81/136 | | n/N = 48/87 | |
| Exon 9 | Placebo | 3.7 (1.9, 9.4) | 0.80 | 3.2 (1.4-5.1) | 0.6 |
| Mutated | | n/N = 45/57 | (0.5-1.28) | n/N = 41/52 | (0.36-0.99) |
| | Investigational | 7.9 (4.2, 10.0) | | 7.9 (4.6-10.5) | |
| | Arm | n/N = 29/51 | | n/N = 25/43 | |
| Exon 20 | Placebo | 5.0 (3.1, 5.8) | 0.83 | 3.2 (1.4-5.2) | 0.46 |
| Mutated | | n/N = 53/71 | (0.56-1.24) | n/N = 46/55 | (0.26-0.80) |
| | Investigational | 5.1 (4.2, 6.8) | | 7.1 (4.6-NA) | |
| | Arm | n/N = 45/72 | | n/N = 18/36 | |
| Wild | Placebo | 4.2 (3.2, 5.0) | 0.79 | 6.8 (4.7-8.6) | 1.02 |
| Type | | n/N = 224/292 | (0.65-0.96) | n/N = 126/188 | (0.79-1.30) |
| | Investigational | 6.9 (4.6, 7.7) | | 6.8 (4.7-8.5) | |
| | Arm | n/N = 187/292 | | n/N = 124/199 | |

Robustness of Data

Overall, the ctDNA subpopulation was consistent with the Full population in terms of patient and disease characteristics, and prior therapies. However, a few potential imbalances were noted between the two treatment arms, which could be presumed to have impacted the assessment of treatment benefit.

To further explore the robustness of the treatment effect observed in the PIK3CA^{mut} by ctDNA subpopulation relative to the PIK3CA W by ctDNA subpopulation, additional supportive analyses were performed.

Multivariate Analysis

Retrospective assessment of the baseline characteristics across the ctDNA PIK3CA^{mut} by ctDNA and PIK3CA W by ctDNA subpopulations identified the following potentially relevant imbalances:
In the PIK3CA^{mut} by ctDNA subpopulation (for buparlisib plus fulvestrant vs. placebo plus fulvestrant):
   Median time from initial diagnosis to study entry: 73.8 vs. 51.3 months
   Visceral disease: 60.9% vs. 68.1% of patients (primarily driven by differences in the proportion of patients with lung metastases [27.6% vs. 37.2%] as a similar percentage of patients reported liver metastases [3% vs. 36.3%])
In the PIK3CA^{WT} by ctDNA subpopulation:
   Median time from initial diagnosis to study entry: 78.5 vs. 63.7 months
   Chemotherapy in metastatic setting: 20.1% vs. 29.8%

The median time to progression after initial diagnosis was longer in the buparlisib plus fulvestrant treatment arm for both the PIK3CA^{mut} and PIK3CA^{WT} subpopulations (and could thus be indicative of potentially more indolent disease). However, the observed difference in the time from initial diagnosis to study entry was largely negated as:
a. Similar differences were noted for the Full population and all subgroups but these did not translate into clinical benefit of the same magnitude
b. This difference was almost entirely accounted for in the time from initial diagnosis to first recurrence; the disease prognosis (or disease journey) for subsequent treatment outcomes appears to be similar for all patients after their first recurrence
c. Median time to progression on the most recent therapy was similar for both treatment arms suggesting comparable disease state at the time of study entry for the PIK3CA^{mut} by ctDNA subpopulation (slight difference in the PIK3CA^{mut} population, i.e. 15.9 vs. 13.6 months).

Given these imbalances, a multivariate Cox regression analysis was performed to obtain covariate-adjusted treatment effect estimates, i.e. adjusted hazard ratios. These adjusted hazard ratios allow an assessment of the robustness of the primary hazard ratio and its sensitivity to potential baseline prognostic factors that were unbalanced in the ctDNA subpopulation. The approach taken was as follows:
Covariate-adjusted treatment effect estimates were obtained based on a multivariate Cox regression model with the following factors: treatment, covariates: visceral disease, time from diagnosis until first recurrence ≥24 months, time from last treatment until progression ≥6 months
Treatment by covariate interactions were explored for visceral disease, time from diagnosis until first recurrence ≥24 months, and time from last treatment until progression ≥6 months. For each covariate, a model including treatment, covariate, and treatment by covariate interaction was considered.

The results from the multivariate Cox analysis did not show evidence of an interaction between treatment and visceral disease, the time from last treatment until progression, or the time from diagnosis until first recurrence as the treatment-covariate interaction term was not statistically significant. The covariate-adjusted treatment effect estimate in the PIK3CA^{mut} by ctDNA subpopulation was consistent with the unadjusted hazard ratio (HR 0.56; 95% CI: 0.39, 0.81).

In conclusion, these data suggest that the imbalances observed in baseline characteristics did not influence the treatment effect estimate.

1. A method of treating a patient having a cancer, comprising administering a therapeutically effective amount of a PI3K inhibitor selected from the group consisting of 5-(2,6-di-morpholin-4-yl-pyrimidin-4-yl)-4-trifluoromethyl-pyridin-2-ylamine and its hydrochloride salt and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide) to the patient on the basis of the patient having been determined to have in their circulating tumor DNA (ctDNA) a PIK3CA mutation.

2-4. (canceled)

5. The method according to claim 1, wherein the PI3K inhibitor is (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide).

6. The method according to claim 1, wherein the cancer is selected from a cancer of the lung and bronchus; prostate; breast; pancreas; colon and rectum; thyroid; liver and intrahepatic bile duct; hepatocellular; gastric; glioma/glioblastoma; endometrial; melanoma; kidney and renal pelvis; urinary bladder; uterine corpus; uterine cervix; ovary; head and neck; multiple myeloma; esophagus; acute myelogenous leukemia; chronic myelogenous leukemia; lymphocytic leukemia; myeloid leukemia; brain; oral cavity and pharynx; larynx; small intestine; non-Hodgkin lymphoma; melanoma; and villous colon adenoma.

7. The method according to claim 1, wherein the cancer is selected from breast cancer and head and neck cancer.

8. The method according to claim 1, wherein the cancer is breast cancer.

9. A method of treating a patient having a cancer with a PI3K inhibitor selected from the group consisting of 5-(2,6-di-morpholin-4-yl-pyrimidin-4-yl)-4-trifluoromethyl-pyridin-2-ylamine and its hydrochloride salt and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide), comprising:
selecting the patient for treatment with said PI3K inhibitor on the basis of the patient having been determined to have in their circulating tumor DNA (ctDNA) a PIK3CA mutation; and thereafter, administering a therapeutically effective amount of said PI3K inhibitor to the patient.

10. A method of treating a patient having a cancer with a PI3K inhibitor selected from the group consisting of 5-(2,6-di-morpholin-4-yl-pyrimidin-4-yl)-4-trifluoromethyl-pyridin-2-ylamine and its hydrochloride salt and (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide), comprising:
a) assaying a blood or a plasma sample comprising ctDNA from the patient having breast cancer for the presence of a PIK3CA mutation in the ctDNA; and
b) administering a therapeutically effective amount of said PI3K inhibitor to the patient on the basis of that patient having been determined to have a PIK3CA mutation.

11. The method of claim 1, wherein the PIK3CA mutation includes a mutation in exon 1, 2, 5, 7, 9 and/or 20 in the PIK3CA gene.

12. The method of claim 11, wherein the PIK3CA mutation comprises one or more of the following mutations R263Q, R277W, R278W, K331E, K333N, K333N, G353D, E1093K, C1258R, E1624K, E1633K, E1634G, Q1636K, H3140K, H3140R, H3140L, and/or H3139Y.

13. The method of claim 1, wherein the presence of the PI3KCA mutation in ctDNA is detected by a technique selected from the group consisting of polymerase chain reaction (PCR), reverse transcription-polymerase chain reaction (RT-PCR), TaqMan-based assays, direct sequencing, or Beaming.

14. (canceled)

15. The method according to claim 9, wherein the PI3K inhibitor is (S)-Pyrrolidine-1,2-dicarboxylic acid 2-amide 1-({4-methyl-5-[2-(2,2,2-trifluoro-1,1-dimethyl-ethyl)-pyridin-4-yl]-thiazol-2-yl}-amide).

16-23. (canceled)

24. The method according to claim 9, wherein the cancer is selected from a cancer of the lung and bronchus; prostate; breast; pancreas; colon and rectum; thyroid; liver and intrahepatic bile duct; hepatocellular; gastric; glioma/glioblastoma; endometrial; melanoma; kidney and renal pelvis; urinary bladder; uterine corpus; uterine cervix; ovary; head and neck; multiple myeloma; esophagus; acute myelogenous leukemia; chronic myelogenous leukemia; lymphocytic leukemia; myeloid leukemia; brain; oral cavity and pharynx; larynx; small intestine; non-Hodgkin lymphoma; melanoma; and villous colon adenoma.

25. The method according to claim 9, wherein the cancer is selected from breast cancer and head and neck cancer.

26. The method according to claim 9, wherein the cancer is breast cancer.

27. The method according to claim 1, wherein the breast cancer is HR+, HER2-negative locally advanced or metastatic breast cancer.
